(19) United States
(12) Patent Application Publication
(10) Pub. No.: US 2015/0265825 A1
(43) Pub. Date: Sep. 24, 2015

(54) COMBINED SONIC AND IONTOPHORETIC SKIN CARE DEVICE

(71) Applicant: L'Oreal, Paris (FR)

(72) Inventors: Zane Bowman Allen Miller, Seattle, WA (US); Kenneth A. Pilcher, Seattle, WA (US); James Christopher McInnes, Seattle, WA (US)

(73) Assignee: L'Oreal, Paris (FR)

(21) Appl. No.: 14/222,327

(22) Filed: Mar. 21, 2014

(51) Int. Cl. A61M 37/00; A61M 35/00; A47K 7/04; A61N 1/32

(52) U.S. Cl. CPC A61M 37/0092; A61N 1/325; A61M 35/003; A47K 7/043

(57) ABSTRACT

The present disclosure seeks to address the primary problem of long application durations to deliver an effective dose of an active ingredient. The devices combine mechanical motion with electrically induced flux (e.g., iontophoresis). The combination of techniques in a single device can reduce the treatment time required to deliver the active ingredient in an effective dose, or deliver a larger dose over the same duration of application.

### BACKGROUND

The non-invasive, transdermal delivery of both low and high molecular weight therapeutic agents has long been the goal of researchers around the world. Even ignoring the desire to eliminate invasive delivery techniques, such as hypodermic injection, that are painful and can open the skin to infection, there are many cases where the condition being treated is in the skin itself. Whether the condition is pathological or cosmetic, in most cases the standard method of care is the application of a topical agent that is intended to be absorbed directly through the horny layer of the skin and penetrate to the epidermis or dermis layers where treatment occurs. However, the normal function of skin runs precisely counter to this kind of treatment. Keratinized cells and multiple lipid bilayers in the stratum corneum present a significant barrier, not only to infectious or harmful agents, but also to great many cosmetic treatments and external drug preparations. The result is that for most topical preparations, if flux of the active agent across the stratum corneum happens at all, it happens at a very slow rate, requiring longer treatment times and higher dosages.

To overcome this barrier in a noninvasive fashion, several techniques have been developed to increase transdermal flux. Generally, they fall into two categories. The first category attempts to increase flux by increasing the permeability of the skin. This group includes adding chemicals to the topical formulation that cause the skin to allow greater penetration, encapsulating the active agent in lipophilic molecules that pass easier through the lipid bilayers, and techniques such as electroporation and sonophoresis that cause temporary, reversible micropores in the lipid bilayers. The second category attempts to increase transdermal flux by creating an energy gradient that causes the active ingredient to travel down that gradient across the stratum corneum (and deeper, if desired) via some kind of radiation pressure. This group includes iontophoresis and magnetophoresis, which use electrical and magnetic fields respectively. These techniques are described below in more detail.
Iontophoresis—Iontophoresis is a technique that uses a small electric charge to deliver an agent through the skin. By creating an electric field between two electrodes contacting the skin, active transport of an ion (charged molecule) through the skin can be achieved. The ion in an appropriate formulation is repelled by the source electrode that carries the same charge as the ion, driving it through the stratum corneum and towards the return electrode. Many active ingredients in skin care have ionic forms, so iontophoresis can improve penetration of these ingredients into the epidermis.
Electroporation—Electroporation is a technique for temporarily increasing the permeability of lipid bilayers such as those present in the stratum corneum and in individual skin cell membranes. By creating short bursts of relatively high voltage electric fields, reversible micropores can be formed in lipid bilayers, briefly increasing permeability. In skin, this effect causes the formation of both intra and intercellular pathways for the delivery of active ingredients.
Magnetophoresis—Magnetophoresis is a technique that uses a gradient magnetic field to drive diamagnetic (NOT ferromagnetic) molecules away from the source of the field (the magnet). Diamagnetic materials create an opposed magnetic field in the presence of an applied magnetic field, meaning that a diamagnetic material will be repulsed by the applied magnetic field. Magnetophoresis has been used to enhance transdermal flux of certain drugs that exhibit diamagnetism.
Sonophoresis—Sonophoresis is a technique for increasing transdermal flux of active ingredients with the application of low frequency ultrasound, coupled to the skin through a cream or gel. The increase in kinetic energy causes reversible disruptions in the intracellular lipid bilayers in the stratum corneum, effectively allowing larger, more complex or hydrophilic molecules to penetrate the skin.

Several approaches to increasing flux combine two or more of the techniques listed above. U.S. Pat. No. 7,427,273 (incorporated herein by reference in its entirety), for instance, describes a system that incorporates both sonophoresis and iontophoresis.

Current methods for noninvasive, transdermal and intradermal delivery of active therapeutic agents have two downsides: the lack of depth control and the long duration of application required to deliver an effective dose (low flux). The long application times are further exacerbated by low perception and comfort thresholds for electrical current experienced by most subjects. Although higher levels of current would be considered safe, subjective discomfort can cause a lower limit to be imposed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a dermal formulation delivery device is provided. In one embodiment the device includes:

(a) a device head comprising a source of oscillatory or reciprocating mechanical motion at a sonic frequency; and

(b) an electrical system, comprising:
(i) a first electrode, disposed on the device head and configured to be in electrical communication with a subject's skin;
(ii) a second electrode, configured to be in electrical communication with the subject's skin; and
(iii) a power source in electrical communication with the first electrode and the second electrode;

wherein the electrical system is configured such that the first electrode and the second electrode are configured to be in electrical communication with separate locations of the subject's skin simultaneously during operation.

In another aspect, a method of delivering a charged active molecule in a formulation at a location on a subject's skin is provided. In one embodiment, the method includes:

(a) topically applying the formulation to the location;

(b) directing at the location a source of oscillatory or reciprocating mechanical motion; while concurrently

(c) applying at the location an applied electrical current configured to move the charged active molecule into the subject's skin.

In another aspect, a method of increasing a rate of delivery of a charged active molecule in a formulation at a location on the subject's skin by increasing an electrical current perception threshold of the subject is provided. In one embodiment, the method includes:

(a) topically applying the formulation to the location;

(b) directing at the location a source of oscillatory or reciprocating mechanical motion; while concurrently

(c) applying at the location an applied electrical current configured to move the charged active molecule into the subject's skin, wherein the applied electrical current exceeds a non-stimulated perception threshold current of the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C schematically illustrate representative devices in accordance with the disclosed embodiments;

FIG. 2A is a perspective drawing of a representative device in accordance with the disclosed embodiments;

FIG. 2B is a perspective photograph of an exemplary device in accordance with the disclosed embodiments;

FIG. 3A is a perspective drawing of a representative brush device in accordance with the disclosed embodiments;

FIGS. 3B and 3C are perspective photographs of exemplary devices in accordance with the disclosed embodiments;

FIG. 4 is a photograph of three exemplary device heads in accordance with the disclosed embodiments; and

FIG. 5 is a graphical illustration comparing absorption of a fluorescent compound into skin using known techniques and those of the disclosed embodiments.

### DETAILED DESCRIPTION

The present disclosure seeks to address the primary problem of long application durations to deliver an effective dose of an active ingredient (also referred to herein as an “active” or an “active molecule”) into the skin. In this case, the embodiments described below add new methods (and related devices) to the list of techniques for improving transdermal flux, which when combined with another flux enhancing method, in this case iontophoresis, can reduce the treatment time required to deliver an effective dose or deliver a larger dose over the same duration of application.

The disclosed embodiments include a handheld, personal appliance that stimulates the skin and increases the penetration rate or flux of cosmetic or therapeutic molecules to the epidermis. The disclosed embodiments seek to address the problem of long application times for absorption by adding an additional method of delivering disruptive energy to the skin system. In this case, the primary energy modality applied is mechanical, inducing small strain in the skin to increase permeability via improved transappendageal pathways. The secondary energy modality applied is electrical, inducing electroosmotic flux of a charged active transport across the stratum corneum. This results in an electromechanical device that enables multiple skincare actions. One is to increase delivery and penetration of active ingredients into the epidermis. Another is to reduce the concentration of an active ingredient in a formulation required to achieve the same results for a given application time. In certain embodiments and therapy profiles, a third is improved cleansing over manual washing.

The disclosed embodiments also allows for increased flux by increasing the subject's perception threshold (i.e., the electrical current amount at which the subject perceives the current, thereby causing discomfort). The addition of mechanical oscillating or reciprocating motion to the source electrode universally increases the amount of current delivered before the subject is able to actively perceive a nervous response to the electrical current, usually doubling the amperage. The cause for this is twofold. Firstly, the vibrating sensations from the oscillating electrode are somewhat intense and can mask or override the tingling sensation from the electrical current. Secondly when most subjects feel the current, it rapidly goes from being perceived to being uncomfortable. This appears to happen because a particularly low resistance electrical path will from in a single spot, at which point all of the current is concentrated on a smaller area. By constantly moving the electrode through oscillation, that pathway takes longer to form, or more likely, does not form at all.

While the various aspects of the present disclosure are presented with examples related to skin care, it will be appreciated that the disclosed examples are illustrative in nature, and therefore, should not be construed as limited to skin care applications. It should therefore be apparent that these various aspects of the present disclosure have wide application to any dermal delivery.

In one aspect, a dermal formulation delivery device is provided. In one embodiment the device includes:

(a) a device head comprising a source of oscillatory or reciprocating mechanical motion at a sonic frequency; and

(b) an electrical system, comprising:
(i) a first electrode, disposed on the device head and configured to be in electrical communication with a subject's skin;
(ii) a second electrode, configured to be in electrical communication with the subject's skin; and
(iii) a power source in electrical communication with the first electrode and the second electrode;

wherein the electrical system is configured such that the first electrode and the second electrode are configured to be in electrical communication with separate locations of the subject's skin simultaneously during operation.

The dermal formulation delivery device will now be described in detail.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. In some instances, well-known process steps have not been described in detail in order to not unnecessarily obscure various aspects of the present disclosure. Further, it will be appreciated that embodiments of the present disclosure may employ any combination of features described herein.

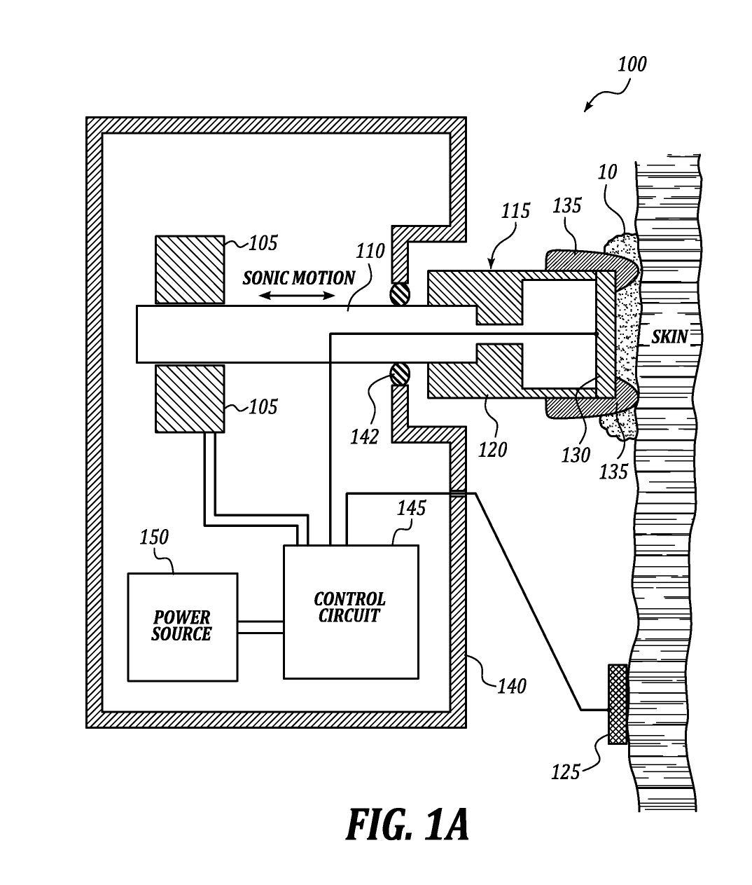

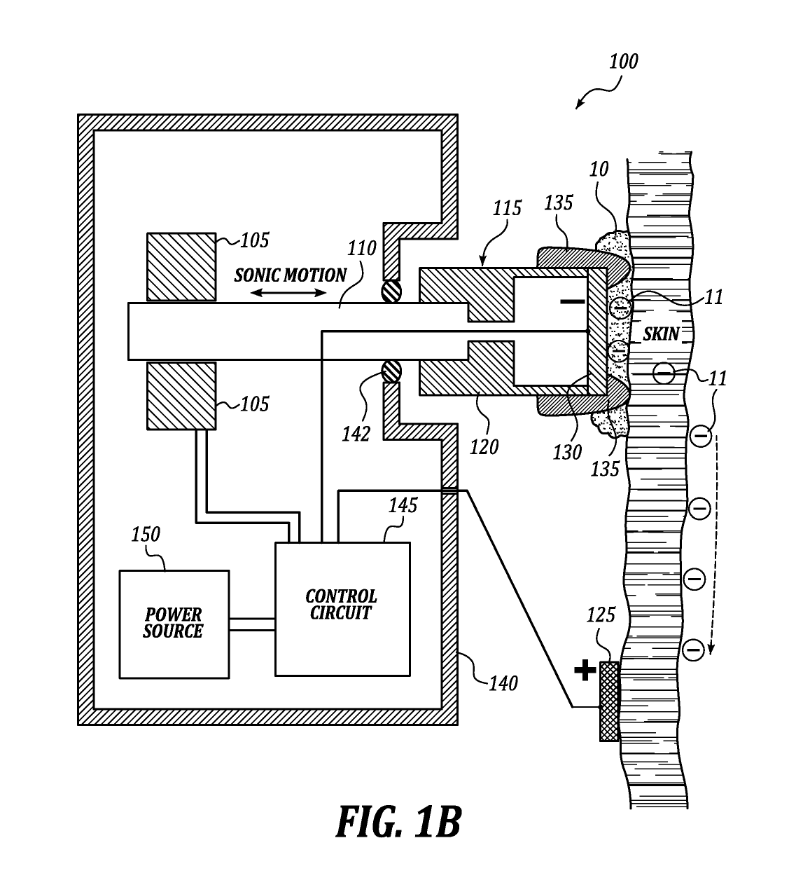

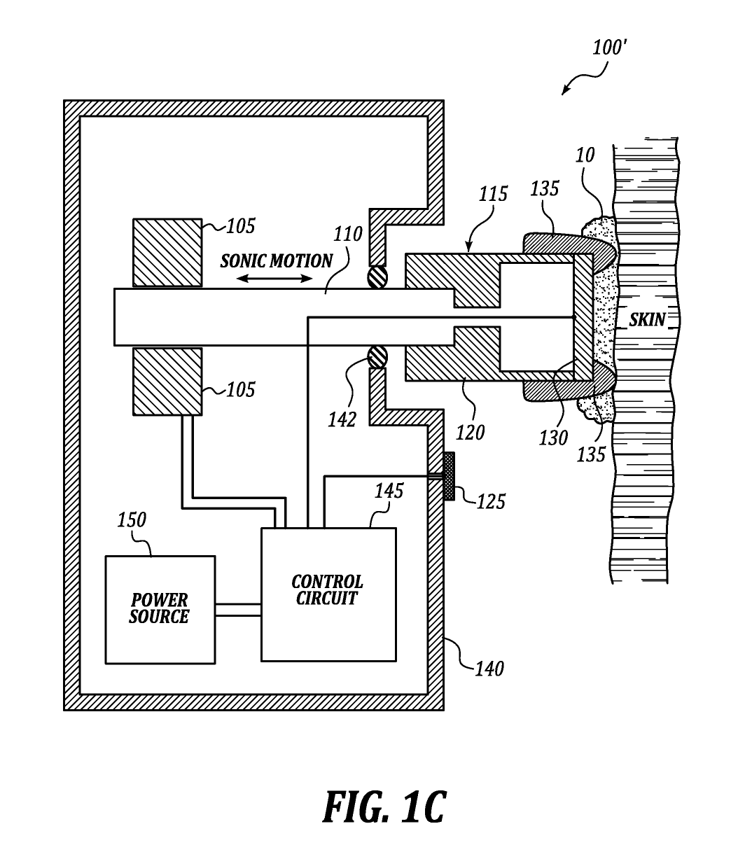

Turning now to FIG. 1A, there is shown one example illustrated in cross section of a dermal formulation delivery device, generally designated 100, formed in accordance with aspects of the present disclosure. The device 100 includes a sonic source 105 (reciprocating or oscillatory motion) directed towards the skin of the subject at a location. The sonic motion provided by the device 100 aids in transdermal delivery of a formulation 10 (e.g., a skin care formulation) into the skin by inducing small strain in the skin to increase permeability via improved transappendageal pathways.

The sonic source 105 provides sonic motion to the location on the skin via a shaft 110 operatively connected to a device head 115. As illustrated, the device head 115 terminates in a concave soft contact member 135 configured to gently contact the skin with a pocket suitable for containing the formulation 10 therebetween.

In one embodiment, the soft contact member 135 is formed from a materials such as an elastomeric material such as silicone rubber, soft enough to avoid discomfort or injury to the skin but firm enough to maintain its shape and impart sufficient sonic energy. Other exemplary materials can also be used, such as natural rubber, butyl rubber, and polyurethane.

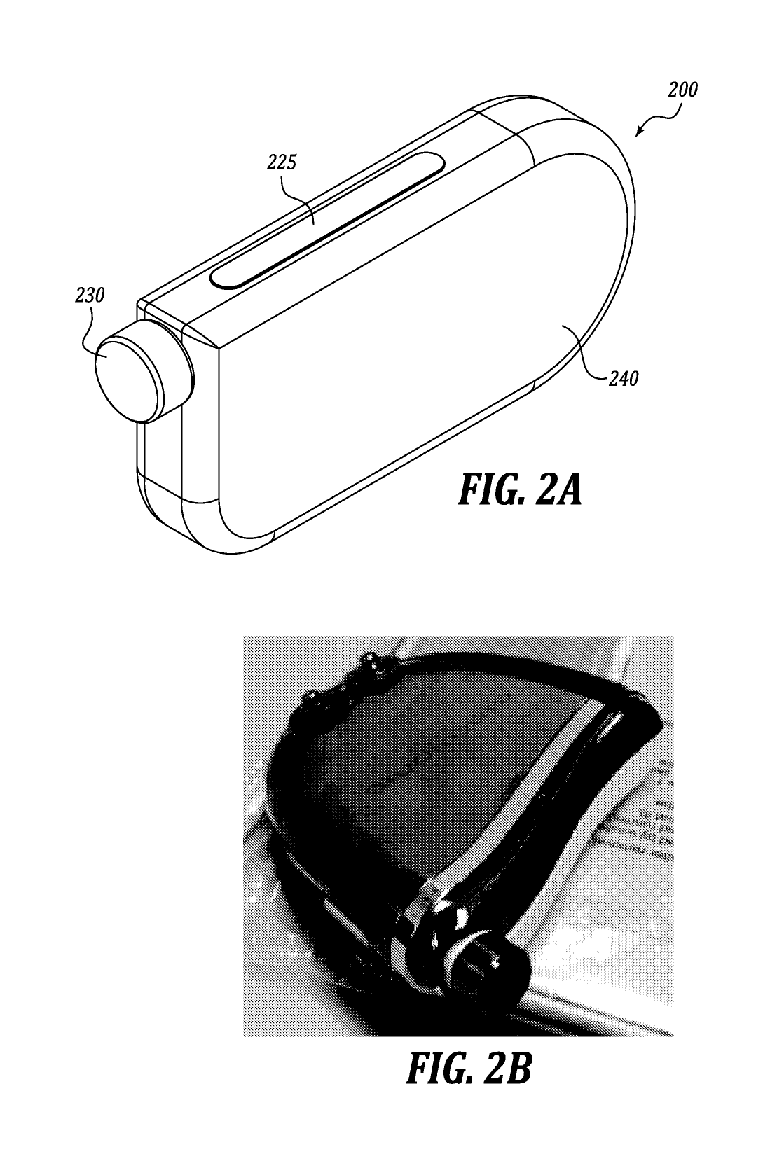

In other embodiments, however, no contact member 135 is present, such as in the embodiments illustrated in FIGS. 2A and 2B.

In an exemplary mode of operation, the sonic source 105 reciprocates the device head 115 at an amplitude of 0.010 inch to 0.1 inch.

In the representative device of FIG. 1A, a housing 140 provides a grip/handle for a user of the device 100. In one embodiment the housing 140 (“body”) is sized and configured to be held in a hand. The housing 140 contains the sonic source 105 and partially encloses the shaft 110, which passes from the housing 140, towards the device head 115 via an aperture fitted with a gasket 142 configured to allow oscillating motion of the shaft 110 while sealing the inner chamber of the housing 140.

The housing 140 further contains a control circuit 145 (e.g., a printed circuit board, field-programmable gate array, ASIC, etc.) configured to operatively control the sonic source 105 and electrodes 125 and 130. As will be described in more detail below, the sonic and electrical signals can be coordinated with regard to timing, intensity, and/or duration. These are controlled by the control circuit 145. A power source 150 is contained within the housing 140 and powers the control circuit 145, the sonic source 105, and the electrodes 125 and 130.

Electrical current is applied to the subject's skin via a first electrode 130 on the device head and a second electrode 125 in a separate location on the subject's skin. By placing the two electrodes 125 and 130 apart on the skin, an electric field gradient is generated that can drive charged actives into the skin (e.g., via iontophoresis). Synergistic effects can be achieved by using iontophoresis to drive the active into the skin at the location which is being impacted by the mechanical force of the device head 115 increasing skin permeability.

Besides iontophoresis, the device can also be configured for transcutaneous electrical nerve stimulation (TENS). For instance, for pain management or to induce muscle contractions as part of electrical muscle stimulation (EMS).

The first electrode 130 is contained within a head housing 120 of the device head 115. In one embodiment the first electrode 130 terminates in a concave portion configured to retain a formulation in between the device head and the subject's skin. In one embodiment the first electrode 130 head terminates in a flat portion. In one embodiment the first electrode 130 is selected from the group consisting of stainless steel, chrome and nickel. In one embodiment the first electrode 130 comprises a material selected from the group consisting of silver (Ag), silver chloride (AgCl), and a combination thereof.

In one embodiment the first electrode 130 is replaceable with a consumable Ag—AgCl conductive layer. Such an electrode has both pure Ag and AgCl salt on the same electrode surface. This allows the same source electrode to be used, regardless of whether the active ingredient in the formulation is positively or negatively charge. The electrode is “consumable” because of the surface chemistry between the electrode and water. In order to generate a galvanic current with something like a chrome, steel or carbon electrode, the source of the counter ion is hydrolysis. This results in a pH change in the solution, which can cause problems, including that the pH will rise above the pKa of the target molecule, basically stopping iontophoresis. With the Ag—AgCl electrode pair, the electrode itself becomes the source of counter ions (Cl—). However, once all of the Ag atoms have been bonded to Cl ions or the AgCl matrix has been depleted of Cl ions, then the electrode has been consumed and needs to be replaced.

The second electrode 125 can be made from a material that is the same or different than that of the first electrode 130. In the embodiment illustrated in FIG. 1A, the second electrode is connected to the control circuit 145 via a wire or cord that runs through the wall of the housing 140. It will be appreciated that the illustrated embodiment is only exemplary and any scheme for electronically connecting the second electrode 125 to the control circuit 145, including via intermediate electrical devices or connections, is contemplated.

An alternative configuration of the second electrode 125 is embodied in the device 100′ illustrated in FIG. 1C, wherein the second electrode 125 is mounted on, or embedded within, the housing 140. This configuration is somewhat simplified in that no additional wire connecting the second electrode 125 to the housing 140 is required. However, this device 100′ must be operated by the subject, due to the requirement that the second electrode contacts the subject's skin away from the location of the first electrode. Therefore, when using the device 100′, the circuit between electrodes 125 and 130 is completed through the body of the subject from the location on the subject's skin where the device contacts (e.g., on the arm or face) to the subject's hand that grips the device 100′ and contacts the second electrode 125. Accordingly, in one embodiment the second electrode 125 is attached to the housing 140 (i.e., body) and configured to be in electrical communication with the subject's skin via a hand of the subject holding the housing 140.

The device 100 of FIG. 1A can be operated by a technician who is not the subject because both electrodes 125 and 130 can be configured to contact the skin of the subject without the subject holding the device 100 in the hand.

Power requirements for the device will depend on the ultimate function of the device. With appropriate circuitry a battery pack can typically handle the required load, or wall power can be used.

Referring now to FIG. 1B, when the device 100 is in operation, the sonic motion acts upon location on the skin to provide mechanical force. Simultaneously, a voltage is applied across the electrodes 125 and 130 with a conductive path, starting (1) at the first electrode 130 (illustrated as a negative electrode); traveling (2) through the formulation 10, which contains (in this embodiment) negatively charged actives 11; traveling (3) into the subject's skin; and the circuit is completed at the second electrode 125 (illustrated as a positive electrode). In the illustrated embodiment of FIG. 1B the device 100 produces an iontophoretic force on the charged actives 11 that drives them into the skin as they attempt to migrate towards the positive electrode.

The mechanical force of the sonic motion reduces resistance of the skin to the actives 11 and allows them to penetrate deeper into the skin/tissue than if no sonic mechanical force were used (assuming the same applied electrical current).

While FIG. 1B illustrates negatively charged actives 11 driven towards a positive second electrode 125 distant from the location where the formulation 10 is applied, it will be appreciated that other configurations are contemplated, such as a positively charged active that is driven towards a negatively charged second electrode 125. The device can be configured to provide the correct polarity and current to drive a particular active into the skin, whether the active is positively or negatively charged.

Specific aspects of the various embodiments of the disclosed devices will now be discussed.

### Mechanical Motion

The device head includes a source of oscillatory or reciprocating mechanical motion at a sonic frequency. As used herein, the term “oscillatory” refers to motion that is a regular periodic motion bi-directionally about a neutral position in a plane largely parallel to the skin surface. As used herein, the term “reciprocating” refers to motion that is a regular periodic motion bi-directionally about a neutral position in a plane largely perpendicular to the skin surface. These two terms are not mutually exclusive and both motions can be combined create more complex motions.

Regardless of the type of motion used, the motion is restricted to displacements within the elastic range of skin. That is, the displacements that cause strain remain within the range where elastin is the dominant load bearing protein in the skin matrix. Beyond the elastic range of skin, the skin would plastically and permanently deform or simply tear.

Reciprocating Motion.

In one embodiment the source of mechanical motion is a source of reciprocating motion. The source of reciprocating motion produces motion at sonic frequencies. In one embodiment, the first source has a reciprocation rate of less than 1 kHz. In one embodiment, the first source has a reciprocation rate of less than 200 Hz. In one embodiment, the first source has a reciprocation rate of greater than 10 Hz. In one embodiment the source of reciprocating motion has a reciprocation rate of 20 to 200 Hz. In one embodiment the source of reciprocating motion has a reciprocation rate of 110 to 135 Hz.

In one embodiment, the source of reciprocating motion is selected from the group consisting of a motor, a pneumatic device, and a piezoelectric device. Such sources of reciprocating motion at sonic frequencies are known to those of skill in the art and can be implemented in the disclosed device accordingly.

An exemplary device for providing reciprocating sonic movement is the Opal (Clarisonic, Redmond, Wash.), which is illustrated in a form modified from the commercial device, in FIG. 2A. In this device 200, the device head 230 creates strain on the skin immediately adjacent to the area of the skin that is in contact with the applicator. U.S. Patent Application Publication No. 2009/0306577, incorporated herein by reference in its entirety, describes an exemplary reciprocating device (such as the Opal) that can apply a sonic motion through a device head 230. This action increases skin permeability by temporarily flexing and enlarging dermatoglyphs, paracellular spaces or transappendageal pathways such as hair follicles and sweat glands, which in turn increases dermal delivery. The action of the device head 230, which is substantially perpendicular to the skin, also acts to drive a formulation into the epidermis. This driving force occurs regardless of the formulation composition.

The device 200 of FIG. 2A has a first electrode that forms the surface of the device head 230, and a second electrode 225 that is mounted on the device body 240 so as to contact the skin of the subject operating the device 200. In this regard, the device 200 is similar to the device 100′ of FIG. 1C.

FIG. 2B shows an example of a prototype exemplary reciprocating, transverse shear prototype. This embodiment accepts interchangeable source electrodes and required an external iontophoresis current source.

Oscillatory Motion.

In one embodiment the source of mechanical motion is a source of oscillating motion. The source of oscillating motion produces motion at sonic frequencies. In one embodiment, the first source has an oscillation rate of less than 1 kHz. In one embodiment, the first source has an oscillation rate of less than 200 Hz. In one embodiment, the first source has an oscillation rate of greater than 10 Hz. In one embodiment the source of oscillating motion has an oscillation rate of 20 to 1000 Hz. In one embodiment the source of oscillating motion has an oscillation rate of 20 to 80 Hz.

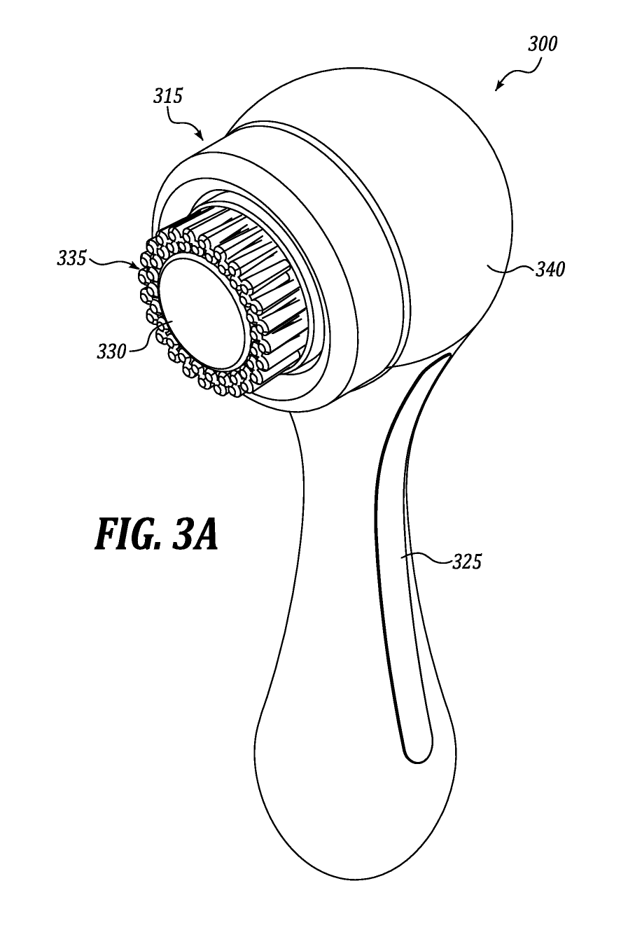

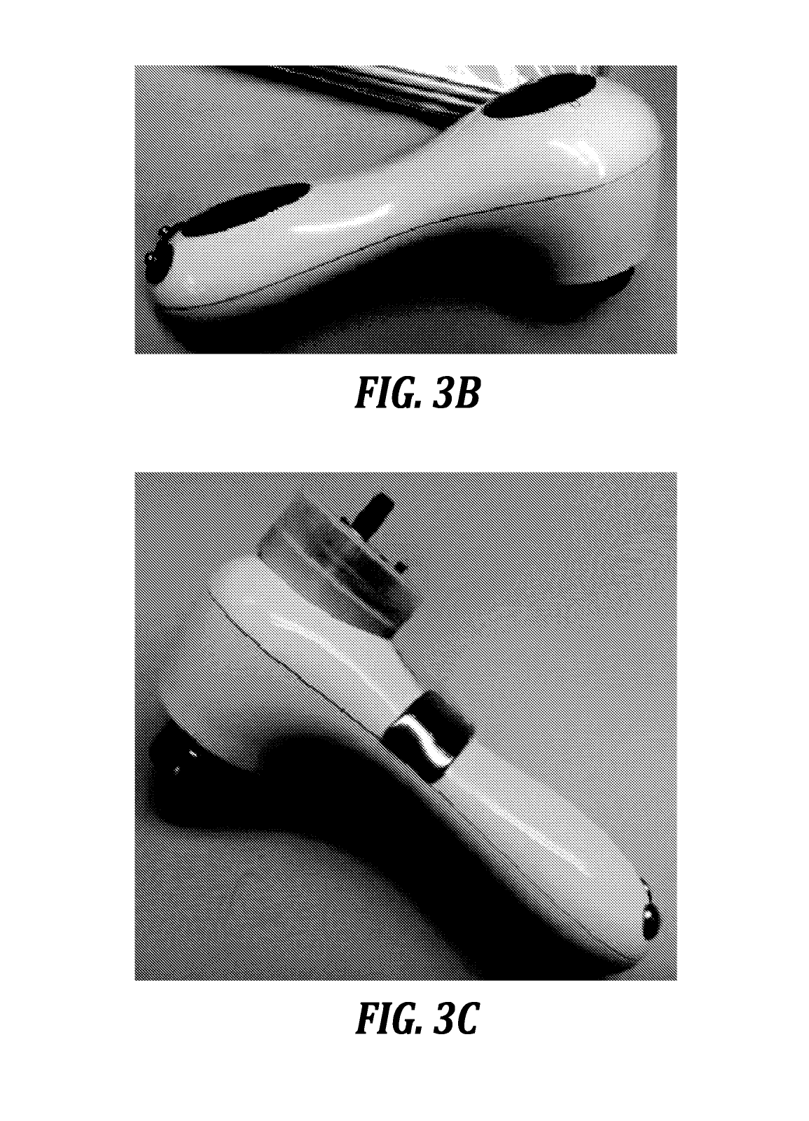

An exemplary device for providing reciprocating sonic movement is the Clarisonic Brush (Clarisonic, Redmond, Wash.), which is illustrated in a form modified from the commercial device, in FIG. 3A. In this device 300, a brush head 315 is mounted to a body 340 that includes a motor or other source of oscillatory motion that is translated to the brush head 315. The brush head 315 includes a bristle field 335 integrated with a first electrode 330, in such a way that skin strain occurs in or immediately adjacent to the area of the skin that is in contact with the first electrode 330. Or the first electrode 330 can be smooth without bristles. A second electrode 325 is integrated into the body 340 to complete the circuit through the skin of the subject holding the device 300.

U.S. Pat. No. 7,320,691 (incorporated herein by reference in its entirety) describes the optimal frequency and strain. This action increases skin permeability by temporarily flexing and enlarging transappendageal pathways such hair follicles and sweat glands, which in turn increases transdermal flux.

FIG. 3B shows an example of a first generation oscillating, shear strain handle without an integrated iontophoresis current generator. This embodiment works with interchangeable source electrodes of different shapes and materials and has return electrodes of chrome plated ABS.

FIG. 3C shows a second generation oscillating, shear strain embodiment with an external iontophoresis current generating circuit that receives power from the device batteries. This embodiment also accepts interchangeable source electrodes.

In one embodiment the device head further comprises a non-conductive element configured to contact the subject's skin.

In one embodiment the non-conductive element comprises a brush with bristle tufts configured to contact the subject's skin simultaneously with the first electrode.

In one embodiment the bristles are arranged surrounding the first electrode.

In one embodiment the bristles are configured to contact the subject's skin within an electric field generated between the first electrode and the second electrode.

### Electrodes

Source Electrodes.

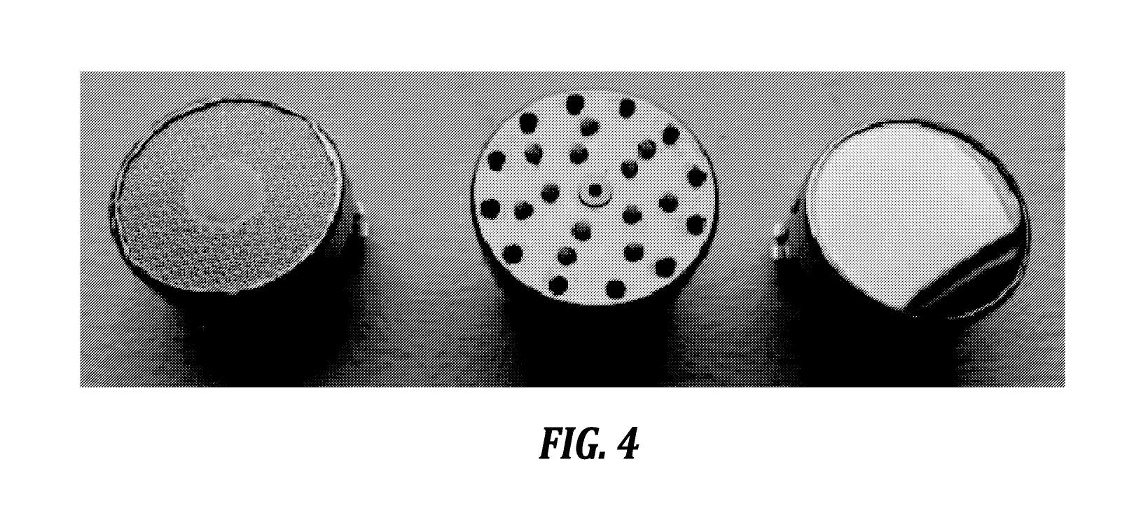

The “source” electrodes are the electrodes attached to the device head (e.g., first electrodes 130, 230, and 330). Multiple source electrodes were developed for both the oscillating, shear strain embodiment and the reciprocating transverse strain embodiment. Some examples are shown in the FIGURES. FIG. 4 shows shear strain electrodes made in different conductive materials with varying textures.

Design considerations for the source electrodes include maximizing surface area and conductivity, frictional characteristics, mass and inertial characteristics, and material compatibility and safety.

Return Electrodes.

The “return” or second electrode (e.g., 125, 225, or 325) are separate from the location on the subject's skin where the first electrode contacts as part of the device head. As disclosed above, the second electrode may be attached to the subject's skin via a wire from the body of the device or may be integrated into the body of the device so as to make contact when the device is held by the subject.

Design considerations for the return electrodes include maximizing surface area and conductivity as well as material compatibility and safety.

### Iontophoresis

Electrode

Electrode design for iontophoresis focuses on achieving the largest contact area possible for maximum flux. The materials chosen should produce minimal changes in pH at the skin surface and avoid hydrolysis as well as inhibit corrosion for long life. Depending on the ultimate function of the device, the shape and placement of the source and return electrodes could differ. For iontophoresis, a single source electrode is embedded in the bristle field with the return electrode contacting the skin somewhere else away from the treatment site. U.S. Pat. No. 7,069,073 (incorporated herein by reference in its entirety) teaches that the return electrode can be located in the handle. If a membrane enclosed formulation reservoir is employed, it could be designed to be pliable in order to conform to sharp contours in the skin (such as around the nose).

Electrical Characteristics

Previous research has shown that the maximum iontophoretic current used for humans should not exceed 0.5 mA/cm². Voltages between 10 V-30 V have been shown to result in reduced resistance in the skin.

Electrical characteristics for the disclosed devices include constant DC current in one embodiment. In another embodiment an AC signal can be used (e.g., pulsed waveform of 1 kHz with a 60% duty cycle).

In one embodiment the electrical system is configured to maintain a constant current density when used on the subject, wherein the constant current density is maintained by periodic adjustment to an applied voltage. In one embodiment the electrical system is configured to provide a current density of up to 0.5 mA/cm².

As disclosed in the Examples below, an unexpected effect of the combination of mechanical motion and iontophoresis is that the mechanical motion serves to “mask” the applied electrical current used for iontophoresis. Because of this masking the amount of current that can be applied before a subject perceives the electrical current (reaching the perception threshold current, thus producing discomfort) is increased. This increase in current serves to further drive the iontophoresis and better dermal delivery of the active can be achieved. In the experimental results presented below an average increase of subject perception threshold current was greater than 100% when comparing the non-stimulated perception threshold to the mechanically stimulated perception threshold current. Accordingly, in one embodiment the electrical current used on a subject is increased by 100% or more compared to the subject's non-stimulated perception threshold current.

### Formulation

Integral to the effective function of the device is an appropriately formulated topical solution that contains the ionic form of active ingredients desired. Referring to FIGS. 1A-1C, the formulation 10 is positioned between the subject's skin and the first electrode 130.

The formulation has a pH and ion concentration (both active and competing) to maximize the flux of the active into the epidermis.

In one embodiment, the formulation is applied to the device head prior to contacting the subject's skin. In one embodiment, the formulation is applied to the location on the subject's skin prior to the steps of directing the sonic motion and directing the ultrasonic motion. In one embodiment, the formulation improves action between the location and the first source of oscillatory motion. In one embodiment, the formulation improves action between the location and the second source of oscillatory motion. In one embodiment the device comprises a formulation reservoir configured to deliver the formulation between the subject's skin and the first electrode.

In one embodiment the formulation is a formulation configured for a use selected from the group consisting of skin care, skin cleansing, skin purification, skin exfoliation, skin desquamation, massage, cellulite, thinning, make up, and depigmentation.

In one embodiment the formulation comprises a charged species of an active ingredient selected from the group consisting of analgesics, anesthetics, anti-inflammatories, anticoagulants, therapeutic peptides, oligonucleotides, and cosmetic actives.

In one embodiment the formulation comprises an active ingredient selected from the group consisting of aspirin, atropine, caffeine, epinephrine, hyaluronic acid, insulin, L-ascorbic acid and deriviatives thereof, lidocaine, hbFGF, ribonuclease, and RNAse T1.

In one embodiment the formulation comprises an active ingredient selected from humectants and moisturizing ingredients, and anti-aging actives.

Humectants and moisturizing ingredients may be in particular glycerol and its derivatives, urea and its derivatives, especially Hydrovance marketed by National Starch, lactic acid, hyaluronic acid, AHA, BHA, sodium pidolate, xylitol, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestlé under the name NutraLipids, a C-glycoside derivative such as those described in WO 02/051828 and in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestlé, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

In one embodiment the formulation comprises a depigmenting agent.

Depigmenting agents include vitamin C and its derivatives and especially vitamin CG, CP and 3-O ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive.

Preferred depigmenting agents include vitamin C and its derivatives and especially vitamin CG, CP and 3-O ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

In one embodiment the formulation comprises an anti-wrinkle active.

As used herein, the term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines.

Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof.

Examples of such compounds are: adenosine and its derivatives and retinol and its derivatives such as retinol palmitate, ascorbic acid and its derivatives such as magnesium ascorbyl phosphate and ascorbyl glucoside; tocopherol and derivatives thereof such as tocopheryl acetate, nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof.

As adenosine derivatives include especially non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside.

Other derivatives include adenosine receptor agonists including adenosine adenosine phenylisopropyl (“PIA”), 1-méthylisoguanosine, N6-cyclohexyladenosine (CHA), N6-cyclopentyladenosine (CPA), 2-chloro-N6-cyclopentyladenosine, 2-chloroadenosine, N6-phényladénosine, 2-phénylaminoadénosine, MECA, N 6-phénéthyladénosine, 2-p-(2-carboxy-ethyl) phenethyl-amino-5′- -N-ethylcarboxamido adenosine (CGS-21680), N-ethylcarboxamido-adenosine (NECA), the 5′(N-cyclopropyl)-carboxamidoadénosine, DPMA (PD 129.944) and metrifudil.

Other adenosine derivatives include compounds that increase the intracellular concentration of adenosine, such as erythro-9-(2-hydroxy-3-nonyl) adenine (“EHNA”) and iodotubercidine.

Others adenosine derivatives include salts and alkyl esters.

By C-glycoside derivative, is meant in particular the compounds described in EP-1345919 of the formula (I): R-SIX (A) wherein S represents a monosaccharide or a polysaccharide up to 20 units sugar in pyranose and/or furanose form and of L and/or D, said mono- or polysaccharide having at least one hydroxyl function that is necessarily free and/or optionally one or more optionally protected amine functions, the bond S—CH 2-X represents a bond of C-anomeric nature, X represents a group selected from: —CO—, —CH(OH)—, —CH(NR1R2)-, —CHR′—, —C(═CHR′)—, R represents a linear or branched alkyl, perfluoroalkyl chain, hydrofluoroalkyl, saturated or unsaturated cycloalkyl ring, cycloperfluoroalkyl cyclohydrofluoroalkyl comprising from 1 to 18 carbon atoms, phenyl or benzyl, said channel, said ring or said radical can be optionally interrupted by one or more heteroatoms selected from oxygen, sulfur, nitrogen, silicon, and optionally substituted with at least one radical chosen from—Or′1, —SR′s, —NR″R′2′1, —COOR″2, —CONHR′″2, —CN, halogen, perfluoroalkyl, hydrofluoroalkyl and/or at least one cycloalkyl radical, aryl, optionally substituted heterocyclic, R′, R1, R2, identical or different, have the same meaning as that given for R, and can also be hydrogen and a hydroxyl radical, R′1, R′2, R″1, R″2, R′″1, R′″2, identical or different, represent a hydrogen atom, hydrogen, a radical selected from alkyl, hydroxy, perfluoroalkyl and/or hydrofluoroalkyl, linear or branched, saturated or unsaturated, comprising from 1 to 30 carbon atoms.

An active promoting skin microcirculation assets acting on the cutaneous microcirculation can be used to avoid dulling of the complexion and/or improve the appearance of the eye contour, in particular to reduce dark circles.

It may for example be selected from an extract of maritime pine bark as Pycnogenol® from Biolandes, manganese gluconate (GIVOBIO GMn Seppic), an extract of Ammi Visnaga as Visnadine of Indena, lupine extract (Eclaline Silab), the coupling protein hydrolyzed wheat/palmitic acid with palmitic acid as Epaline 100 Carilene Laboratories, the extract of bitter orange blossom (Remoduline Silab) vitamin P and its derivatives such as methyl-4 monoéthanoate esculetol sodium sold under the name Permethol Sephytal, extracts of ruscus, brown guinea, ivy, sweet clover and ginseng, caffeine nicotinate and derivatives thereof, lysine and its derivatives such as Asparlyne Solabia, a black tea extract as Kombuchka Sederma; rutin salts: an extract of the alga Corallina officinalis, such as marketed by Codif, and mixtures thereof.

Preferred agents for promoting the cutaneous microcirculation, we include caffeine, extract of bitter orange blossom, a black tea extract, rutin salts, an extract of the alga Corallina officinalis.

In one embodiment the formulation comprises an active ingredient that addresses oily skin. These actives can be sebo-regulating or antiseborrhoeic agents capable of regulating the activity of sebaceous glands. These include: retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Sérobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of ‘meadowsweet (Spiraea ulmaria) such as that sold under the name Cytobiol Ulmaire by sociétéLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL® ECI by Sasol; trialkyl citrate (C14-C15) sold under the name COSMACOL® ECL by Sasol—10-hydroxydecanoic acid, including mixtures acid-hydroxydecanoic October 25, sebacic acid and 1,10-decandiol such as that sold under the name Acnacidol BG by Vincience and—mixtures thereof.

Antiseborrheic active ingredients include: benzoyl peroxide, vitamin B6 (or pyridoxine), 30—zinc salts such as zinc gluconate, zinc pyrrolidonecarboxylate (or zinc pidolate), the zinc lactate, zinc aspartate, zinc carboxylate, zinc salicylate, zinc cysteate—extracts of meadowsweet (Spiraea ulmaria), such as that sold under the name Sebonormine® by the company Silab—extracts of the alga Laminaria saccharina, such as that sold under the name Phlorogine by Biotechmarine—mixtures of extracts of salad burnet root (Sanguisorba officinalis/Poterium officinale), rhizomes of ginger (Zingiber officinalis) and cinnamon bark (Cinnamomum cassia), such as that sold under the name Sebustop by Solabia—extract clove such as that sold under the name Clove extract powder by Maruzen—filtrates lactic such as that sold under the name Normaseb by Sederma protein—extracts of meadowsweet (Spiraea ulmaria) such as that sold under the name Cytobiol Ulmaire by Libiol;—sebacic acid, sold especially in the form of a sodium polyacrylate gel under the name Sebosoft by Sederma—glycine grafted onto an undecylenic chain, such as that sold under the name Lipacide UG OR by Seppic—the detrialkyle citrate (C12-C13) sold under the name COSMACOL ECI by Sasol; trialkyl citrate (C14-C15) sold under the name COSMACOL ECL by Sasol—the 10-hydroxydecanoic acid, and especially mixtures of 10-hydroxydecanoic acid, of sebacic acid and of 1,10-decanediol, such as that sold under the name Acnacidol BG by Vincience—and mixtures thereof.

Preferably, the anti-seborrhoeic active agent is chosen from: zinc salts such as zinc gluconate, zinc pyrrolidonecarboxylate (or zinc pidolate), zinc lactate, zinc aspartate, carboxylate zinc, zinc salicylate, zinc cysteate, and preferably pyrrolidonecarboxylate zinc (or Pidolate zinc) or zinc salicylate—the clove extract such as that sold under the name Clove extract powder by Maruzen—glycine grafted onto an undecylenic chain, such as that sold under the name Lipacide UG OR by SEPPIC—trialkyl citrate (C12-C13) sold under the name COSMACOL ECI by Sasol trialkyl citrate (C14-C15) sold under the name COSMACOL 5 ECL by Sasol—and mixtures thereof.

Antiseborrhoeic active is, for example, present in an amount ranging from 0.1 to 10% by weight, preferably 0.1 to 5% by weight, and preferably from 0.5% to 3% by weight, relative to the total weight of the formulation.

### Active Molecule Delivery Methods

In another aspect, a method of delivering a charged active molecule in a formulation at a location on a subject's skin is provided. As discussed previously, the disclosed embodiments can be used to deliver coordinated mechanical motion and electrical current to a location on a subject's skin in order to produce a synergistic effect that delivers a charged active molecule into the skin in greater amounts than either of the techniques individually.

In one embodiment, the method includes:

(a) topically applying the formulation to the location;

(b) directing at the location a source of oscillatory or reciprocating mechanical motion; while concurrently

(c) applying at the location an applied electrical current configured to move the charged active molecule into the subject's skin.

In one embodiment the applied electrical current is provided by a first electrode and wherein the formulation remains in between the subject's skin and the first electrode.

In one embodiment the formulation is applied to the first electrode or a reservoir on the electrode prior to the formulation contacting the subject's skin.

In one embodiment the formulation is applied to the subject's skin prior to the formulation contacting the first electrode.

In one embodiment the applied electrical current exceeds a non-stimulated perception threshold current of the subject.

In one embodiment the applied electrical current is less than a mechanically stimulated perception threshold current of the subject.

In one embodiment the source of oscillatory or reciprocating mechanical motion and the electrical current are provided by a dermal formulation delivery device comprising:

(a) a device head comprising a source of oscillatory or reciprocating mechanical motion at a sonic frequency; and

(b) an electrical system, comprising:
(i) a first electrode, disposed on the device head and configured to be in electrical communication with a subjects skin;
(ii) a second electrode, configured to be in electrical communication with the subject's skin; and
(iii) a power source in electrical communication with the first electrode and the second electrode;

wherein the electrical system is configured such that the first electrode and the second electrode are configured to be in electrical communication with separate locations of the subject's skin simultaneously during operation.

This type of device has been described in detail throughout this application.

In another aspect, a method of increasing a rate of delivery of a charged active molecule in a formulation at a location on the subject's skin by increasing an electrical current perception threshold of the subject is provided. In one embodiment, the method includes:

(a) topically applying the formulation to the location;

(b) directing at the location a source of oscillatory or reciprocating mechanical motion; while concurrently

(c) applying at the location an applied electrical current configured to move the charged active molecule into the subject's skin, wherein the applied electrical current exceeds a non-stimulated perception threshold current of the subject.

In one embodiment the applied electrical current is less than a mechanically stimulated perception threshold current of the subject.

As described previously, the mechanical motion can mask the applied electrical current and allows for a higher perception threshold current, thereby allowing more current to be used and iontophoresis improved accordingly.

The following examples are included for the purpose of illustrating, not limiting, the described embodiments.

### Examples

### Experimental Platforms

Experimental platforms were constructed for both the oscillating, shear strain embodiment and the reciprocating, transverse strain embodiment were also constructed that connected to external function generators and amplifiers, allowing for variable frequency and amplitude during testing. FIGS. 2A and 2B show exemplary shear strain devices and FIGS. 3A-3C show exemplary transverse shear devices.

### Increased Flux

An initial experiment was conducted to explore and quantify the effectiveness of combining oscillating mechanical shear strain of the skin with iontophoresis for improving penetration of an active ingredient into the skin.

A solution of fluorescein, water and a hydroethylcellulose thickener was applied to properly prepared pig skin samples and then treated for 120 seconds using one of five methods:
1. No treatment (i.e., passive diffusion).
2. Iontophoresis using a smooth, chrome-plated, ABS source electrode at 35V.
3. Mechanical only using a chrome-plated, ABS electrode with embedded bristles run at 150 Hz and 8 degrees of amplitude.
4. Iontophoresis combined with mechanical strain using the smooth electrode at 35V, oscillating at 150 Hz and 8 degrees of amplitude.
5. Iontophoresis combined with mechanical strain using the bristled electrode at 35V, oscillating at 150 Hz and 8 degrees of amplitude.

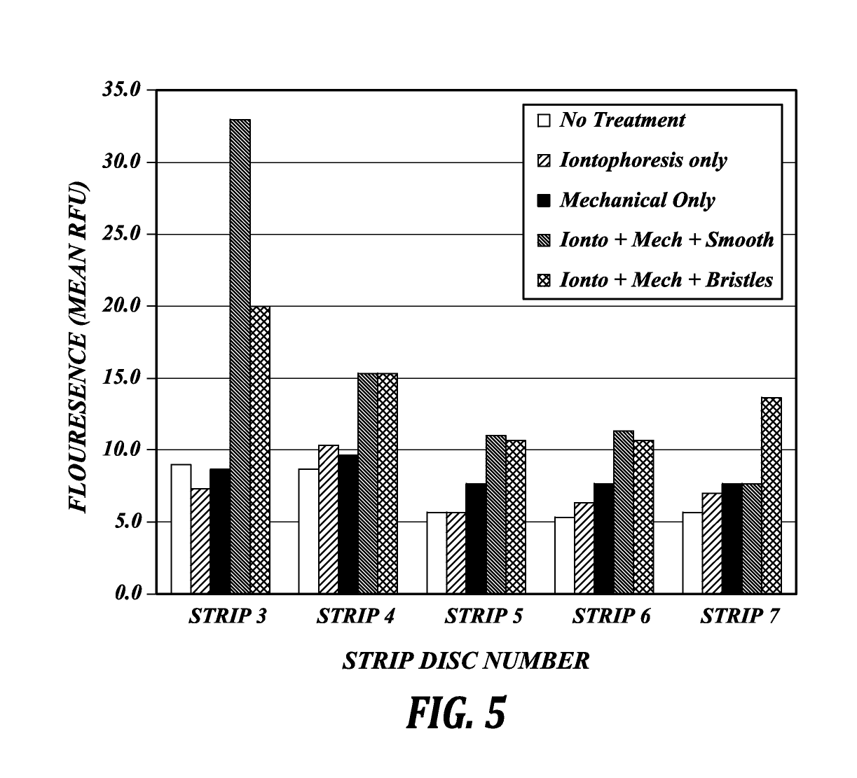

For each treatment method, n=3. Three skin samples were prepared and one test of each treatment method was performed on each sample. After treatment, excess solution was removed with water and the test site was dried with a gauze pad. Using CuDerm D-Squame tape strip sampling discs, seven consecutive samples were collected at each site. Per standard practice, the first two strips at each test site were discarded. The remaining five discs and a blank were affixed to the bottom of a 6-well polycarbonate plate and analyzed for relative fluorescence using a plate reader. The aggregated results are displayed in FIG. 5.

As seen in FIG. 5 these preliminary results indicate a synergistic effect on penetration between the mechanical action applied to the skin and the electrical potential applied to the formulation. There is an increased availability in the skin at each level, also indicating a greater depth of penetration.

### Increased Perception Threshold

An initial experiment was conducted to explore and quantify the effectiveness of combining oscillating mechanical shear strain of the skin with iontophoresis for increasing the minimum current delivered to for it to be perceived by a subject.

Six (n=6) subjects of varying age and gender used a device similar to that of FIGS. 3A-3C coupled with a smooth, chrome plated electrode to deliver an electric current via an aqueous gel to the forearm, first with a static electrode and then with the electrode oscillating at 170 Hz. Current was controlled using a current limited DC power supply with a maximum output voltage of 30 V. Because the current delivered is a function of the applied voltage and the electrical resistance of the subject, a high enough current was not able to be generated to pass the perception threshold in certain subjects. In the event that the maximum output voltage was reached before crossing a subject's perception threshold, the final current in mA was recorded. In the event that the current output went above the 0.5 m A/cm² recommended limit, the experiment was terminated.

TABLE 1
PERCEPTION THRESHOLD RESULTS

| | Perception Threshold (mA) | | | |
|---|---|---|---|---|
| | Non-Stimulated; | Stimulated; | Percent | |
| Subject | Oscillation: Off | Oscillation: On | Increase | Note |
| 1 | 1.6 | 3.6 | 125% | Max voltage |
| | | | | reached |
| 2 | 0.2 | 0.58 | 190% | |
| 3 | 1.2 | 2.5 | 108% | |
| 4 | 0.6 | 1.5 | 150% | |
| 5 | 1.6 | 5 | 213% | Experiment |
| | | | | stopped |
| 6 | 3 | 3.5 | 17% | Max voltage |
| | | | | reached |

Table 1 shows the results of the perception threshold experiment. Ignoring results where a conclusive threshold could not be achieved, there was still an average of a 150% increase in current delivered before crossing a subject's perception threshold.

While illustrative embodiments have been illustrated and described, it will be appreciated that various changes can be made therein without departing from the spirit and scope of the invention.

The embodiments of the invention in which an exclusive property or privilege is claimed are defined as follows:

1. A dermal formulation delivery device, comprising:
(a) a device head comprising a source of oscillatory or reciprocating mechanical motion at a sonic frequency; and
(b) an electrical system, comprising:
(i) a first electrode, disposed on the device head and configured to be in electrical communication with a subject's skin;
(ii) a second electrode, configured to be in electrical communication with the subject's skin; and
(iii) a power source in electrical communication with the first electrode and the second electrode;
wherein the electrical system is configured such that the first electrode and the second electrode are configured to be in electrical communication with separate locations of the subject's skin simultaneously during operation.

2. The dermal formulation delivery device of claim 1, further comprising a body onto which the device head is mounted.

3. The dermal formulation delivery device of claim 2, wherein the body is sized and configured to be held in a hand.

4. The dermal formulation delivery device of claim 3, wherein the second electrode is attached to the body and configured to be in electrical communication with the subject's skin via a hand of the subject holding the body.

5. The dermal formulation delivery device of claim 1, comprising a source of oscillatory motion having an oscillation rate of 20 to 1000 Hz.

6. The dermal formulation delivery device of claim 1, comprising a source of reciprocating motion having a reciprocation rate of 20 to 200 Hz.

7. The dermal formulation delivery device of claim 1, wherein the electrical system is configured to provide a current density of up to 0.5 mA/cm².

8. The dermal formulation delivery device of claim 1, wherein the electrical system is configured to maintain a constant current density when used on the subject, wherein the constant current density is maintained by periodic adjustment to an applied voltage.

9. The dermal formulation delivery device of claim 1, wherein the device head further comprises a non-conductive element configured to contact the subject's skin.

10. The dermal formulation delivery device of claim 9, wherein the non-conductive element comprises a brush with bristle tufts configured to contact the subject's skin simultaneously with the first electrode.

11. The dermal formulation delivery device of claim 10, wherein the bristles are arranged surrounding the first electrode.

12. The dermal formulation delivery device of claim 11 wherein the bristles are configured to contact the subject's skin within an electric field generated between the first electrode and the second electrode.

13. The dermal formulation delivery device of claim 1, wherein the first electrode terminates in a concave portion configured to retain a formulation in between the device head and the subject's skin.

14. The dermal formulation delivery device of claim 1, wherein the first electrode head terminates in a flat portion.

15. The dermal formulation delivery device of claim 1, wherein the first electrode is selected from the group consisting of stainless steel, chrome and nickel.

16. The dermal formulation delivery device of claim 1, wherein the first electrode comprises a material selected from the group consisting of silver (Ag), silver-chloride (AgCl), and a combination thereof.

17. The dermal formulation delivery device of claim 1 wherein the first electrode is replaceable with a consumable Ag—AgCl conductive layer.

18. A method of delivering a charged active molecule in a formulation at a location on a subject's skin, comprising:
(a) topically applying the formulation to the location;
(b) directing at the location a source of oscillatory or reciprocating mechanical motion; while concurrently
(c) applying at the location an applied electrical current configured to move the charged active molecule into the subject's skin.

19. The method of claim 18, wherein the applied electrical current is provided by a first electrode and wherein the formulation remains in between the subject's skin and the first electrode.

20. The method of claim 19, wherein the formulation is applied to the first electrode or a reservoir on the electrode prior to the formulation contacting the subject's skin.

21. The method of claim 19, wherein the formulation is applied to the subject's skin prior to the formulation contacting the first electrode.

22. The method of claim 18, wherein the applied electrical current exceeds a non-stimulated perception threshold current of the subject.

23. The method of claim 22, wherein the applied electrical current is less than a mechanically stimulated perception threshold current of the subject.

24. The method of claim 18, wherein the formulation is a formulation configured for a use selected from the group consisting of skin care, skin cleansing, skin purification, skin exfoliation, skin desquamation, massage, cellulite, thinning, make up, and depigmentation.

25. The method of claim 18, wherein the source of oscillatory or reciprocating mechanical motion and the electrical current are provided by a dermal formulation delivery device that comprises:
(a) a device head comprising a source of oscillatory or reciprocating mechanical motion at a sonic frequency; and
(b) an electrical system, comprising:
(i) a first electrode, disposed on the device head and configured to be in electrical communication with a subjects skin;
(ii) a second electrode, configured to be in electrical communication with the subject's skin; and
(iii) a power source in electrical communication with the first electrode and the second electrode;
wherein the electrical system is configured such that the first electrode and the second electrode are configured to be in electrical communication with separate locations of the subject's skin simultaneously during operation.

26. A method of increasing a rate of delivery of a charged active molecule in a formulation at a location on the subject's skin by increasing an electrical current perception threshold of the subject, the method comprising:
(a) topically applying the formulation to the location;
(b) directing at the location a source of oscillatory or reciprocating mechanical motion; while concurrently
(c) applying at the location an applied electrical current configured to move the charged active molecule into the subject's skin, wherein the applied electrical current exceeds a non-stimulated perception threshold current of the subject.

27. The method of claim 26, wherein the applied electrical current is less than a mechanically stimulated perception threshold current of the subject.
